(19) United States
(12) Patent Application Publication
(10) Pub. No.: US 2024/0215863 A1
(43) Pub. Date: Jul. 4, 2024

(54) TRACKING SEGMENTAL MOVEMENT OF THE HEART USING TENSORS

(71) Applicant: VENTRIPOINT DIAGNOSTICS LTD., Toronto (CA)

(72) Inventors: Desmond HIRSON, Toronto (CA); Alvira MACANOVIC, Toronto (CA); Claudio IRRGANG, Toronto (CA); Mark SIRKIN, Toronto (CA)

(73) Assignee: VENTRIPOINT DIAGNOSTICS LTD., Toronto, ON (CA)

(21) Appl. No.: 18/286,390

(22) PCT Filed: Apr. 12, 2022
(86) PCT No.: PCT/CA2022/050562

§ 371 Date: Oct. 11, 2023

Related U.S. Application Data
(60) Provisional application No. 63/205,658, filed on Apr. 12, 2021.

(51) Int. Cl. A61B 5/11; G06T 7/246

(52) U.S. Cl. CPC A61B 5/1102; G06T 7/246; G06T 2207/10088; G06T 2207/10136; G06T 2207/30048

(57) ABSTRACT

Disclosed is a system, method and/or computer readable medium for use with for imaging the heart and/or segments of the chambers of the heart. Segments from a heart pair (i.e., an ES model and an ED model of the heart) are preferably matched between segments that make up the respective surfaces of the ES and ED models, each segment having a relative angle and displacement. The same segment is present in the two models but at a different position and orientation in three-dimensional space. The displacement of the matching or paired segments indicates the movement of that segment over the heart cycle. In a preferred embodiment, the segments are positioned in the same anatomical structure of the heart, therefore tracking the segments represents the actual anatomic movement. A four-dimensional model may preferably be created from two-dimensional echocardiogram images.

### CROSS-REFERENCE TO RELATED APPLICATIONS

This application is a national phase entry of PCT Patent Application Serial No. PCT/CA2022/050562 filed on Apr. 12, 2022, which claims priority to the U.S. Provisional Patent Application Ser. No. 63/205,658 filed on Apr. 12, 2021, both of which are incorporated by reference herein.

### TECHNICAL FIELD

The present invention relates generally to a system, method and/or computer readable medium for monitoring the heart. In particular, the present invention relates to a system, method and/or computer readable medium for tracking segmental movement of the heart using tensors.

### BACKGROUND

In the prior art, different solutions have been developed to depict how the heart wall changes over a cardiac cycle. The prior art attempts, however, may have been limited to devices that show the change in volume and the movement of the heart walls using various methods of tracking.

Prior art research in cardiology has been focused on preserving left ventricular function of the heart because studies have established its importance in determining prognosis for survival. For many years, the right ventricle of the heart may have been ignored because investigators thought it was simply a passive conduit directing blood from the body back to the lungs. Researchers have identified that depressed right ventricular function can influence survival in patients with congenital heart disease and in other conditions affecting the left or right ventricle or both. Studies have confirmed the importance of maintaining right ventricular function for all diagnoses.

Heart function is typically measured by analyzing cardiac images. There are several available imaging modalities in the prior art. The physician chooses the modality based on both medical and practical considerations. Ultrasound (i.e., echocardiogram) is preferred for follow-up studies because it is inexpensive, does not expose the patient to ionizing radiation, is non-invasive and therefore safe, and is under the jurisdiction of cardiology. These advantages must be weighed against limitations of echocardiograms, including: a) 15% of patients have image quality too poor for diagnosis due to chest anatomy characteristics; b) in the remaining patients image quality is influenced by the training and experience of the sonographer (technician acquiring the images). Cardiologists also perform angiography, but the cardiac catheterization procedure exposes the patient to the risks of radiation and having a catheter placed inside the heart as well as being expensive. In addition to selecting the imaging modality, the physician must also select the method for analyzing the images. The left ventricle, with its regular shape, has been compared to an ellipsoid of revolution in most patients except those with congenital heart disease. The approach used for the left ventricle cannot be applied to the right ventricle due to the complex shape of the right ventricle. The left ventricular anatomy of the heart has traditionally been less difficult to model. Conversely, the complexity of the right ventricular anatomy makes it difficult to define. The atria also do not follow a reproducible geometric shape for easy characterization.

In the prior art, cardiologists may have evaluated the volume and function of the right ventricle by visual estimation from 2D echo images. Visual estimation is known to be inaccurate and poorly reproducible but is nevertheless performed due to the unsuitability of other methods.

Cardiac MRI is the gold standard for volumetric cardiac measurements but it is expensive, stressful on the patient, especially in the case of pediatric patients and cannot be performed at the bedside. Most cardiologists may have estimated right ventricular function from 2D echocardiograms by visual inspection (i.e., “eyeballing” the images). They may eschew quantitative methods since these all suffer from high error. The error comes from relating the right ventricle to geometric reference figures that poorly resemble the shape of this chamber. For example, the area-length and multiple-slice methods assume that the right ventricle has an ellipsoid shape or elliptical cross section, respectively. Neither method can accommodate the right ventricle's irregular shape. A third method comparing the right ventricle to a pyramid was found more successful for pediatric patients, but the mean signed error in measuring volume was still high at 16%. Even in patients whose right ventricles fit a geometric shape, accuracy in measuring volume depends on the examiner's ability to locate image planes that yield the maximal area and long axis length measurements. In addition, there are no good landmarks in the right ventricle to help the sonographer find and image the same anatomy on follow-up studies. These problems are magnified when dealing with the variable right ventricular shapes seen in congenital heart disease.

3D echocardiogram includes two modes. The first approach is volumetric imaging, whereby tightly spaced images are acquired to generate a solid volume of image data. To perform quantitative analysis from volumetric studies, the data set is cut into a series of parallel image planes. The user then traces the right ventricular contours in each plane and sums the volumes of the “slices”. However, the right ventricle cannot be imaged in its entirety in most teenage or adult patients because it does not fit into the image sector. This problem may persist in the latest model devices even when the extended sector mode is used, which acquires the data set over four cardiac cycles.

Furthermore, the accuracy of volumetric techniques may be inconsistent. In the prior art, accurate results have been reported when imaging cadaver hearts by tracing right ventricular borders in as many as 49 parallel planes. Another study in the prior art may have obtained good accuracy for measuring right ventricular volume at end diastole but not at end systole (i.e., both are required to measure function) even though the right ventricular borders were traced every two millimeters (i.e., 20 borders for a normal-size adult heart). Thus, despite the time-consuming image analysis, the accuracy for volume determination has not been consistent.

The second 3D echocardiogram approach is to acquire the images by routine freehand scanning while recording the spatial location and orientation of the image planes using a tracking system. The advantage of this approach is that the image data can be acquired from whatever combination of acoustic windows and views provides optimal image quality in any given patient. The borders of the heart structures are then traced in multiple views and used to reconstruct the surface of the right ventricle in 3D. The volume of the right ventricle is then computed from the 3D surface. Several techniques have been reported in the prior art with good accuracy, but the right ventricular border may have to be traced in 10-16 image planes. One report described a method that requires tracing the borders of only three images and extrapolating the right ventricular surface between them; however, the required image planes are hard to find except in cadaver hearts. The piecewise smooth subdivision surface method developed by the University of Washington may also require that borders be traced in multiple views. Thus, measuring right ventricular volume by 3D echo is accurate but not clinically feasible because of the difficulty of acquiring the images and/or the time required for analyzing multiple images to trace the borders.

Magnetic resonance imaging (“MRI”) is considered a gold standard, but MRI analysis is performed using the Simpson's method, similar to volumetric 3D echo, and therefore requires time-consuming manual border tracing. MRI equipment is expensive and not generally available.

Computed Tomography (CT) has been touted in the prior art for providing excellent spatial resolution and visualization of coronary calcification and the coronary arteries themselves. However, measurement of ventricular volume from CT images is a topic of research, requires manual border tracing, and is less available than MRI. Furthermore, the heavy radiation dose required for CT imaging precludes its use for serial studies in children, especially because children are more susceptible to the tissue damaging effects of radiation than adults.

Research on Automated Border Detection (“ABD”) of MRI and echo images has been in progress for one to two decades. Some successes have been achieved for delineating the left ventricle. A major barrier to ABD of the right ventricle may be the anatomy of this chamber. Its inner surface is covered by interlocking muscle bundles which produce an irregularly rough surface whose true base is difficult to delineate automatically. Also, the complex shape of the right ventricle has prevented application of models commonly used to assist ABD of the left ventricle.

In the prior art, three dimensional echocardiograms may not be adapted to capture the full volume of the heart organ. The mechanism is based on acoustics and soundwaves that travel through the body at a certain rate—send out soundwaves, wait for the return of the soundwaves and collect the data. More data is typically expected with complex images. Prior art may have used stacked arrays and sent out blasts of sound waves and waited for return. The problem is that there may be a trade-off between temporal and spatial resolution. When a large amount of data is generated, the rate of data collection may need to decrease resulting in a loss of temporal information. If the rate of data collection is increased, the amount of detail collected may need to decrease resulting in a loss of spatial resolution.

Accordingly, it is a problem in the art to develop a way of qualitatively and quantitatively showing the movement of the RV. There is currently no acceptable approach for the RV in the prior art; the RV is still underserved from an analysis point of view. As a result, there may be a need for, or it may be desirable to provide an approach in the three-dimensional modeling of the RV over time and/or cooperating environment that overcomes one or more of the limitations associated with the prior art.

### SUMMARY

The present disclosure provides a system, method and/or computer readable medium for imaging the heart and/or segments of the chambers of the heart. The system, method and/or computer readable medium of the present invention preferably, but need not necessarily, quantifies the movement of the segments (e.g., angle and displacement) from End Diastolic (“ED”) to End Systolic (“ES”) motions of the heart. A heart pair (i.e., an ES model and an ED model of the heart) is preferably broken down into matching segments that make up the respective surfaces. Therefore, the same segment is present in the two models but at a different position and orientation in three-dimensional space. The displacement of the matching or paired segments indicates the movement of that segment over the heart cycle.

In a preferred embodiment, the segments are positioned in the same anatomical structure of the heart, therefore tracking the segments represents the actual anatomic movement. A four-dimensional model may preferably, but need not necessarily, be created from two-dimensional and/or three-dimensional echocardiogram images. In preferable embodiments, there is also capability for different segments to be displayed and reported for all the chambers of the heart. The system, method, and computer readable medium of the present invention is a novel way of qualitatively and quantitatively showing the movement of the RV.

An aspect of the present invention is directed to a method for measuring a change in magnitude and/or direction of a heart chamber during a heart cycle, wherein the method comprises the steps of: (a) collecting a plurality of images associated with the heart chamber during the heart cycle; and (b) operating one or more processors to: (i) generate two or more three-dimensional models of the chamber during the heart cycle using the plurality of images; (ii) overlay a mesh on each of the two or more three-dimensional models of the chamber; (iii) divide the mesh into a plurality of segments; (iv) measure the change in magnitude and/or direction of each of the plurality of segments during the heart cycle; (v) determine one or more vectors for each of the segments based on the change in magnitude and/or direction; and/or (vi) determine one or more tensors based on the one or more vectors for each of the segments; wherein the tensors represent volume and function of the heart chamber during the heart cycle.

Another aspect of the present invention is directed to a method for measuring a change in magnitude and/or direction of movement of a heart chamber during a heart cycle, wherein the method comprises the steps of: (a) collecting a plurality of 2D images associated with the heart chamber during the heart cycle; (b) generating a plurality of segments of the heart chamber during the heart cycle using segment generation and assigning each of the plurality of segments to a surface of an anatomical position associated with the heart chamber; and (c) quantifying an angle and a displacement of each of the plurality of segments assigned to the surface of the anatomical position from an end diastolic to an end systolic phases of the heart cycle to determine the magnitude and/or direction of movement of each of the plurality of segments assigned to the surface of the anatomical position associated with the heart chamber during the heart cycle.

Another aspect of the present invention is directed to the above noted methods further comprising: (d) determining a plurality of vectors for each of the plurality of segments based on the change in magnitude and direction; and (e) determining a tensor data based on the plurality of vectors for each of the segments to demonstrate an incremental change over time of the movement of the heart chamber between the systolic and diastolic phases of the heart cycle. Another aspect of the invention is directed to the above noted method wherein the tensor data include surface area, contraction coefficient, and length of tensor which represents volume and function of the heart chamber during the heart cycle. Another aspect of the invention is directed to the above noted method wherein the tensor data comprises the data set out in Table 1.

Another aspect of the invention is directed to the above noted method wherein images are made using 2D ultrasound, 3D ultrasound or MRI. Another aspect of the invention is directed to the above noted method wherein the tracking of the plurality of segments measures the movement of the walls of the heart chamber. Another aspect of the invention is directed to the above noted method wherein the heart chamber is selected from a RV, RA, LV, LA.

Another aspect of the invention is directed to the above noted method wherein the tracking of the plurality of segments provides information on the viability or health of the heart chamber. Another aspect of the invention is directed to the above noted method further comprising: (f) using a movement algorithm to track the plurality of segments in the ED volume versus the ES volume as determined by a user selecting specific anatomical landmarks of the heart chamber as an input to a KBR algorithm; and (g) calculating the displacement of the matching plurality of segments to determine the movement of the segments over the heart cycle.

Another aspect of the invention is directed to a method for measuring a change in magnitude and/or direction of a heart chamber during a heart cycle, wherein the method comprises the steps of: (a) collecting a plurality of images associated with the heart chamber during the heart cycle; and (b) operating one or more processors to: (i) generate two or more three-dimensional models of the chamber during the heart cycle using the plurality of images; (ii) overlay a mesh on each of the two or more three-dimensional models of the chamber; (iii) divide the mesh into a plurality of segments; (iv) measure the change in magnitude and/or direction of each of the plurality of segments during the heart cycle; (v) determine one or more vectors for each of the segments based on the change in magnitude and/or direction; and/or (vi) determine one or more tensors based on the one or more vectors for each of the segments; wherein the tensors represent volume and function of the heart chamber during the heart cycle.

Another aspect of the invention is directed to the above noted method wherein step (b) further comprises a user selecting a plurality of triangles within the mesh to generate a single segment within the mesh. Another aspect of the invention is directed to the above noted method wherein the triangles are contiguous or non-contiguous triangles.

Other advantages, features and characteristics of the present invention, as well as methods of operation and functions of the related elements of the system, method and/or computer readable medium for optimizing patient rehabilitation, and the combination of steps, parts and economies of manufacture, will become more apparent upon consideration of the following detailed description and the appended claims with reference to the accompanying drawings, the latter of which are briefly described herein below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features which are believed to be characteristic of the system, method and/or computer readable medium according to the present invention, as to their structure, organization, use, and method of operation, together with further objectives and advantages thereof, may be better understood from the following drawings in which presently preferred embodiments of the invention may now be illustrated by way of example. It is expressly understood, however, that the drawings are for the purpose of illustration and description only and are not intended as a definition of the limits of the invention. In the accompanying drawings:

FIG. 1 is a schematic diagram depicting a three-dimensional model of a RV within a heart in accordance with an embodiment of the present invention;

FIG. 2 is a schematic diagram depicting a plurality of tensors for a portion of the heart in accordance with an embodiment of the present invention;

FIG. 3 is a table listing the segments associated with the RV in accordance with an embodiment of the present invention;

FIG. 4 is a table and schematic diagram depicting a mesh of the RV in accordance with an embodiment of the present invention;

FIG. 5 is a schematic diagram depicting segments of the RV in accordance with an embodiment of the present invention;

FIG. 6 is a schematic diagram depicting a segment of the RV with vectors in accordance with an embodiment of the present invention;

FIG. 7 is a schematic diagram depicting a segment of the RV with a tensor in accordance with an embodiment of the present invention;

FIG. 8 is a schematic diagram depicting a side view of the segment of the RV with the tensor of in accordance with an embodiment of the present invention;

FIG. 9 is a schematic diagram depicting a plurality of vectors in accordance with an embodiment of the present invention;

FIG. 10 is a table listing the segments associated with the RV in accordance with an embodiment of the present invention;

FIG. 11 is a schematic diagram depicting the segments of the RV, the tensors associated with the segments, and the vectors associated with each tensor in accordance with an embodiment of the present invention;

FIG. 12 is a schematic diagram depicting the segments of the RV and the tensors associated with the segments in accordance with an embodiment of the present invention;

FIG. 13 is a schematic diagram depicting segments and associated tensors of the RV in accordance with an embodiment of the present invention;

FIG. 14 is a table listing the segments and associated measurements in accordance with an embodiment of the present invention;

FIG. 15 is a schematic diagram depicting the RV with vectors and a partial mesh in accordance with an embodiment of the present invention;

FIG. 16 is a screenshot of a tensor viewing application in accordance with an embodiment of the present invention;

FIG. 17 is a schematic diagram depicting a three-dimensional model of the RV in accordance with an embodiment of the invention;

FIG. 18 is a screenshot of a tensor viewing application in accordance with an embodiment of the present invention;

FIG. 19 is a schematic diagram depicting a mesh of the RV in accordance with an embodiment of the present invention;

FIG. 20 is a screenshot of a tensor viewing application in accordance with an embodiment of the present invention;

FIG. 21 is a schematic diagram of depicting a mesh, segments and vectors of the RV in accordance with an embodiment of the present invention;

FIG. 22 is a screenshot of a tensor viewing application in accordance with an embodiment of the present invention;

FIG. 23 is a schematic diagram of a RV with segments and tensors associated with the segments in accordance with an embodiment of the present invention;

FIG. 24 is a screenshot of a tensor viewing application in accordance with an embodiment of the present invention;

FIG. 25 is a table listing the segments and associated measurements in accordance with an embodiment of the present invention;

FIG. 26 is a schematic diagram of a RV depicting segments, tensors associated with the segments, and the anterior and longitudinal axes in accordance with an embodiment of the present invention;

FIG. 27 is a schematic diagram of a RV depicting segments, tensors associated with the segments, and the septal axis in accordance with an embodiment of the present invention;

FIG. 28 is a screenshot of a tensor viewing application in accordance with an embodiment of the present invention;

FIG. 29 is a screenshot of a tensor viewing application in accordance with an embodiment of the present invention;

FIG. 30 is a screenshot of a tensor viewing application in accordance with an embodiment of the present invention; and

FIG. 31 is a screenshot of a spreadsheet showing time graphed against longitudinal strain in accordance with an embodiment of the present invention.

### DETAILED DESCRIPTION

The description that follows, and the embodiments described therein, may be provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and, in some instances, proportions may have been exaggerated in order to more clearly depict certain embodiments and features of the invention.

The present disclosure may be now described in terms of an exemplary system in which the present disclosure, in various embodiments, would be implemented. This may be for convenience only and may not be intended to limit the application of the present disclosure. It may be apparent to one skilled in the relevant art(s) how to implement the present disclosure in alternative embodiments.

Certain novel features which are believed to be characteristic of a system for a tracking segmental movement of the heart using tensors, which are novel in conjunction with the cooperating environment, according to the present invention, as to their organization, use, and/or method of operation, together with further objectives and/or advantages thereof, may be better understood from the accompanying disclosure in which presently preferred embodiments of the invention are disclosed by way of example. It is expressly understood, however, that the accompanying disclosure is for the purpose of illustration and/or description only and is not intended as a definition of the limits of the invention.

Persons having ordinary skill in the art will understand that knowledge-based reconstruction (“KBR”) provides a method for rapid measurement of a chamber of the heart (e.g., right ventricular volume and function in patients with congenital heart disease). The measurements are preferably, but need not necessarily, made using images generated by either a two-dimensional ultrasound, three-dimensional ultrasound or magnetic resonance imaging device. To perform KBR, a user is preferably but need not necessarily only required to select or place a number of points on the images to mark the position of anatomic landmarks. In a preferred embodiment, a KBR algorithm utilizes knowledge concerning the shape of the human heart and how it typically deforms in various disease states. In a preferred embodiment, knowledge of the three-dimensional heart size and shape is used to reduce the workload that a human would typically need to accurately measure how well a patient's heart is functioning.

Persons having ordinary skill in the art will appreciate that the heart is a mechanical organ that pumps blood through the circulatory system. The heart has two main pumping chambers, the left ventricle, and the right ventricle. The left ventricle generates high pressure to pump blood through the systemic circulation (body). The right ventricle, which pumps blood through the lung, is only required to generate a low pressure. The function of the heart is typically measured in terms of its “ejection fraction”, the proportion of the filled volume that is moved out in each heartbeat. Ejection fraction is calculated as (EDV-ESV)/EDV and expressed as a percentage, where “EDV” is “end diastolic volume” (i.e., volume when the heart is full) and “ESV” is “end systolic volume” (i.e., volume at the end of a contraction). Each ventricle has an inlet valve and an outlet valve. The valves ensure that blood flows only in the forward direction through the heart.

KBR is fast as it takes about two-three minutes per volume measurement. The user preferably, but need not necessarily, provides only a very sparse input of points (i.e., not whole borders). The user can choose the highest quality images to trace those points. In other words, the user is free to work just on the images where each part of the ventricle is best seen.

KBR does not require tracing of whole borders. This may be advantageous as border tracing can be difficult because the images may not always show the entire border clearly; there is typically some part that is fuzzy and hard to identify. Border tracing takes so much time and effort that cardiologists hesitate to trace even one or two borders for a left ventricular volume compared with the eight or more borders that are typically required for a right ventricular volume.

KBR leverages the accuracy achieved from the sparse input by utilizing a knowledge database. The database embodies knowledge of the shape of the right ventricle and how much that shape varies in human disease. The knowledge database constrains the software to produce heart-like reconstructions and to prevent the possible generation of strangely shaped surfaces.

In a preferred embodiment, the system, method and/or computer readable medium of the present invention relies on any modality adapted to image the heart and generate segments for all four heart chambers by applying, for example, a segment generation algorithm. Persons having ordinary skill in the art may appreciate that typical modalities in the prior art include ultrasound and MRI. The segments are inherent in the KBR technology (referenced above and described in U.S. Pat. No. 5,889,524, incorporated herein by reference). In a preferred embodiment, the system, method and/or computer readable medium of the present invention quantifies the movement of the segments (i.e., angle and displacement of each segment) from End Diastolic (“ED”) to End Systolic (“ES”) motion of the heart. The present invention is a novel approach to quantifying and visualizing heart motion. In a preferred embodiment, a movement algorithm determines the ES and ED volumes of the heart by the user selecting specific anatomical landmarks that is used as an input to the KBR engine that relies on a database of heart shapes derived from clinical studies. Each representative heart in the database preferably, but need not necessarily, is associated with a corresponding pair of ES and ED models of the heart. Each heart pair (i.e., ES and ED) is preferably, but need not necessarily, broken down and/or divided up into matching segments that make up the surfaces. Therefore, any given segment is present in the two volumes or models (i.e., an ES and ED pair) but at a different position and orientation in 3-dimensional space. The displacement of the same (or matching) segments preferably, but need not necessarily, indicates the movement over the heart cycle.

In a preferred embodiment, the segments are positioned in the same anatomical position within each heart model (i.e., ES and ED pair) so that the tracking represents and/or approximates the actual anatomic movement. In a preferred embodiment, the vectors may be adapted for regrouping to allow a user (e.g., clinician) to focus on a particular vector by selecting a desired segment. This provides information with respect to the anatomical segments of the heart preferably, but need not necessarily, including additional and important information to the clinician, which may be helpful when dealing with the complexities of various diseases. 4D models can be created from 2D echo images. In contrast to the present tensor technology, the prior art may only calculate the ES and ED volume without measurement or indication of movement which may result in inaccuracies. For example, monitoring only the ES and ED volume will not allow for the determination of whether the movement of a particular portion of the RV wall (i.e., a segment) is compromised. In other words, the movement of a particular segment of the RV wall between the ES and ED volumes can be monitored and/or measured to provide additional information about the heart. Persons skilled in the art will appreciate that the present invention is not limited to the RV and may be applied to other chambers of the heart. There is also the capability for different segments to be displayed and reported for all four chambers of the heart. The present invention is preferably a novel way of qualitatively and quantitatively showing the movement of all four chambers, including the right ventricle (“RV”). There is currently no acceptable approach for determining the movement of the RV in a patient in the prior art.

The system, method and/or computer readable medium of the present invention may preferably, but need not necessarily, provide the following features set out in Table 1.

TABLE 1
Proposed 4D Tensor Software Features

| | How it is | How parameter is | How parameter will |
|---|---|---|---|
| Parameter | calculated/derived | expressed | be displayed |
| Global | Change in length as a | Percentage | Involves view |
| longitudinal strain | proportion to baseline | derived from (L₁- | selection, defining |
| | length; illustrate | L₀) / L₀ × 100 | end-systole, tracing |
| | contraction and temporal | | the myocardium; |
| | dispersion in multiple | | comprised of |
| | myocardial segments. | | magnitude (degree |
| | | | of myocardial |
| | | | deformation) and |
| | | | direction (cardiac |
| | | | axes of myocardial |
| | | | motion) |
| Spatial orientation | Three main spatial | Percentage | Measurement value. |
| for 3D | orientations of myocardial | | |
| | contraction-Four-chamber | | |
| | apical view showing | | |
| | longitudinal displacement | | |
| | from base to apex, and | | |
| | short axis view at the left | | |
| | ventricular papillary | | |
| | muscle level showing | | |
| | radial displacement | | |
| | towards the center and | | |
| | circumferential | | |
| | displacement along side | | |
| | the cardiac structure. | | |
| Pre-set | Segmentation options | See on the 3D | Load a file with |
| segmentation | include 5 segment, 10 | model the | preselected |
| options for left and | segments, 15 segments; | segments that | segmentations. |
| right ventricle | include coronary artery | have been | |
| | territories for apical, mid, | selected. | |
| | and basal | | |
| Manual | User can select from 17 | See on the 3D | Hover over segment |
| segmentation | segments, including basal | model, the | and select. |
| options for left and | interior, basal anteroseptal, | segments that | |
| right ventricle | basal inferoseptal, basal | have been | |
| | inferior, basal | selected. | |
| | inferolateral, basal | | |
| | anterolateral, mid anterior, | | |
| | mid anteroseptal, mid | | |
| | inferoseptal, mid inferior, | | |
| | mid inferolateral, mid | | |
| | anterolateral, apical | | |
| | anterior, apical septal, | | |
| | apical inferior, apical | | |
| | lateral, and apex. | | |
| Strain rate for 3D | Rate of deformation or | Derivative of the | Graph |
| | stretch occurring over | length. | |
| | time. Defined by the | | |
| | formula which is a | | |
| | derivative of the | | |
| | Lagrangian strain | | |
| | equation: | | |
| | $ε {R}_{L} ( t ) = \frac{{dS}_{L} ( t )}{dt} = \frac{1}{{L}_{0}} \frac{dL ( t )}{dt}$ | | |
| | Where, SR_{L} is Lagrangian | | |
| | strain rate. L₀ is the | | |
| | reference length at time t₀ | | |
| | (usually end diastole). | | |
| | Assess temporal | | |
| | differences in the | | |
| | generation of maximal | | |
| | systolic deformation | | |
| | among different | | |
| | myocardial segments | | |
| Surface | Calculate surface area | Percentage | Number for each |
| area/Expansion | when selecting faces on | derived from (S₁- | segment |
| coefficient | the mesh between end- | S₀)/S₀ × 100 | |
| | diastole and end-systole | | |
| Volume-over-time | Curves including peak | mL/s | Graph |
| for left and right | filling and ejection | | |
| ventricle | | | |
| 4D model of left | Pumping of ventricle | N/A | Image of pumping |
| and right ventricle | | | heart chamber. |
| (mesh or solid | | | |
| surface) | | | |
| Vector movement | Derived from tracking of | Orientation and | 3D presentation and |
| | specific segments. | magnitude. | measurement value. |

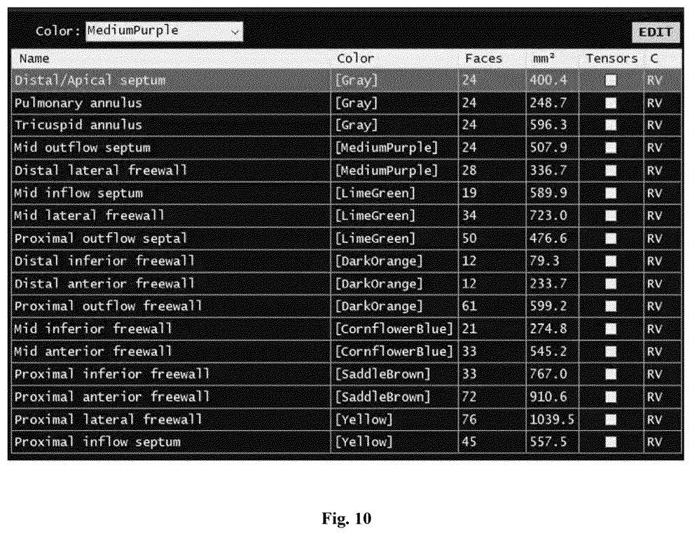

In a preferred embodiment, vector movements (i.e., magnitude and direction) are quantified to model the movement of the heart (including pediatric and adult) as shown, for example, in FIG. 9. Persons having ordinary skill in the art may appreciate that movement of the heart may be compromised by cardiac disease (e.g., hypertension, etc.). Accordingly, the present invention may preferably, but need not necessarily, determine the normal movement of the heart and how the movement of the normal heart differs from a diseased heart (e.g., compromised movement of a portion of the heart wall).

In a preferred embodiment, the present invention is applied to the right ventricle but may also be applied to any other chamber of the heart.

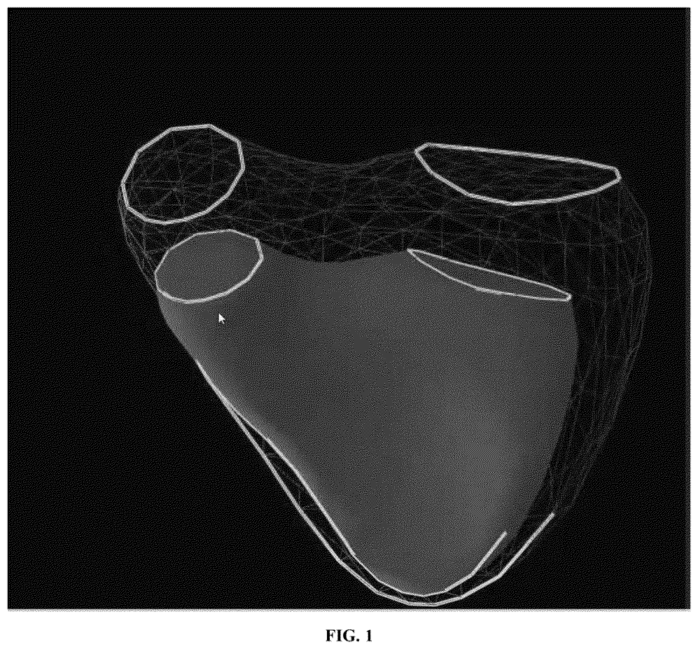

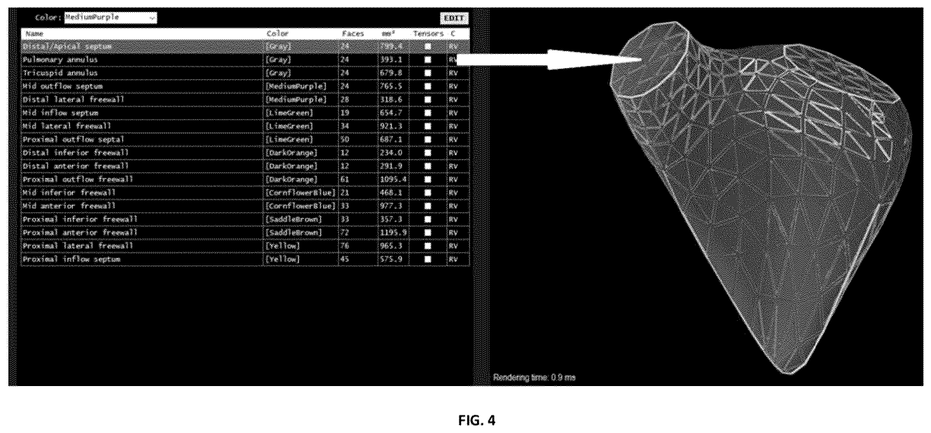

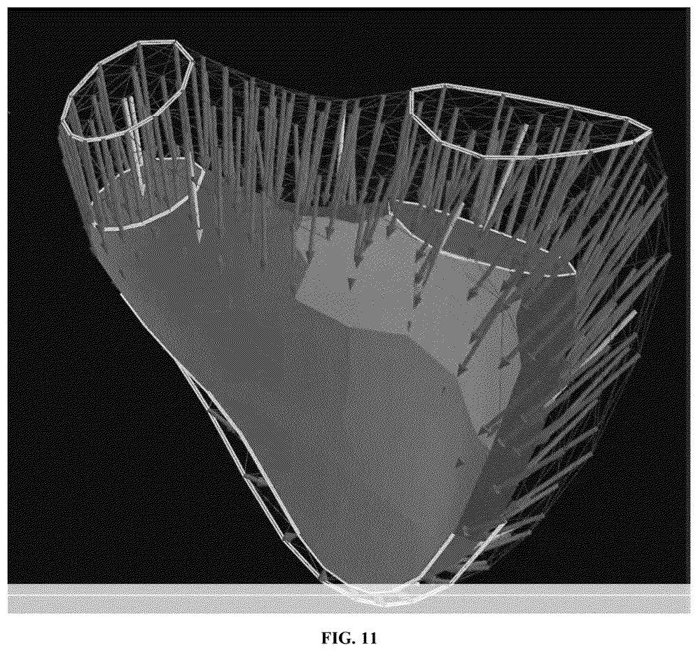

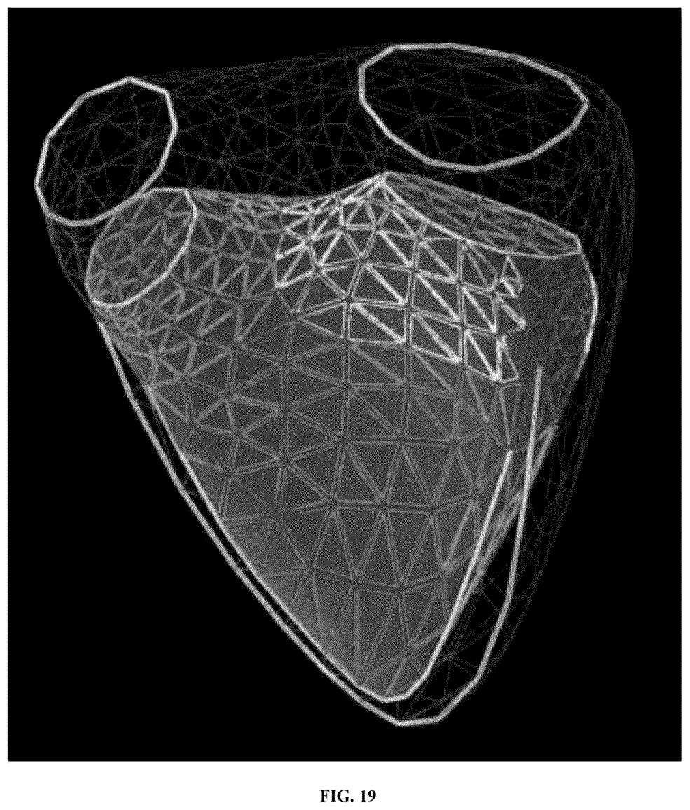

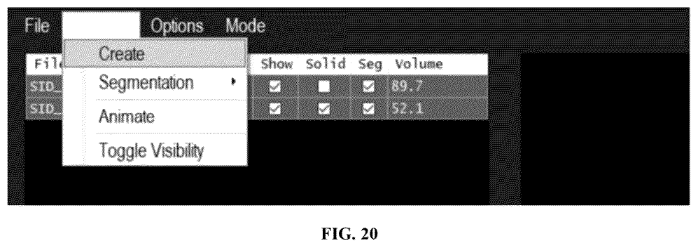

In a preferred embodiment, slices are taken from a two-dimensional and/or three-dimensional image and used to create 3D models as shown, for example, in FIG. 1. A mesh is preferably, but need not necessarily, overlaid the 3D model to define a plurality of discrete areas or openings that are preferably, but need not necessarily, triangular in shape as shown in FIGS. 4 and 19. The mesh may preferably, but need not necessarily, have the same basic structure between models of different hearts or with the same model but at different times within the cycle of a heart. Each mesh may preferably, but need not necessarily, be labeled to facilitate tracking how the mesh changes during the heart cycle as shown in FIGS. 3, 4 and 10. The number of triangles (or discrete areas) is preferably, but need not necessarily, the same between the 3D models of an ES and ED pair to facilitate comparisons. In a preferred embodiment, one mesh can be taken to determine and/or measure the difference from other meshes associated with the heart.

Persons having ordinary skill in the art may appreciate that the left ventricle and right ventricle of the heart have different anatomies which may make it difficult to create 3D models of the chambers. In a preferred embodiment, the present invention allows for the creation of 3D models of the right ventricle and the use of 3D meshes to perform calculations including, for example, determining surface areas and/or volumes.

In comparison, the ultrasound systems of the prior art may not be able to capture the full volume of the one or more chambers of the heart. Traditional ultrasound systems include mechanisms based on acoustics. Sound waves travel through the body at a certain rate. The more complex the image, the more data that is expected. A technical limit to the amount of data that can be transmitted and received. Two-dimensional image systems typically send information in one big blast or signal burst. From a technical perspective, there is a trade-off between temporal resolution (i.e., number of images per time) and spatial resolution (i.e., number of images per area). An increase in data (e.g., increase in number of two-dimensional images) typically requires a trade-off for temporal or spatial resolution. An increase in spatial resolution, decreases temporal resolution. An increase in temporal resolution, decreases spatial resolution.

The present invention preferably, but need not necessarily, facilitates the measurement (e.g., changes in shape and volume of one or more heart chambers) of a beating heart including the steps of: imaging the one or more heart segments; measuring the displacement of the one or more heart segments; and/or measuring the orientation of the one or more heart segments. The displacement and/or angle of each of the one or more heart segments are preferably, but need not necessarily, measured throughout the heart cycle. In a preferred embodiment, the tracking of the one or more heart segments provides information on the viability or health of cardiac tissue.

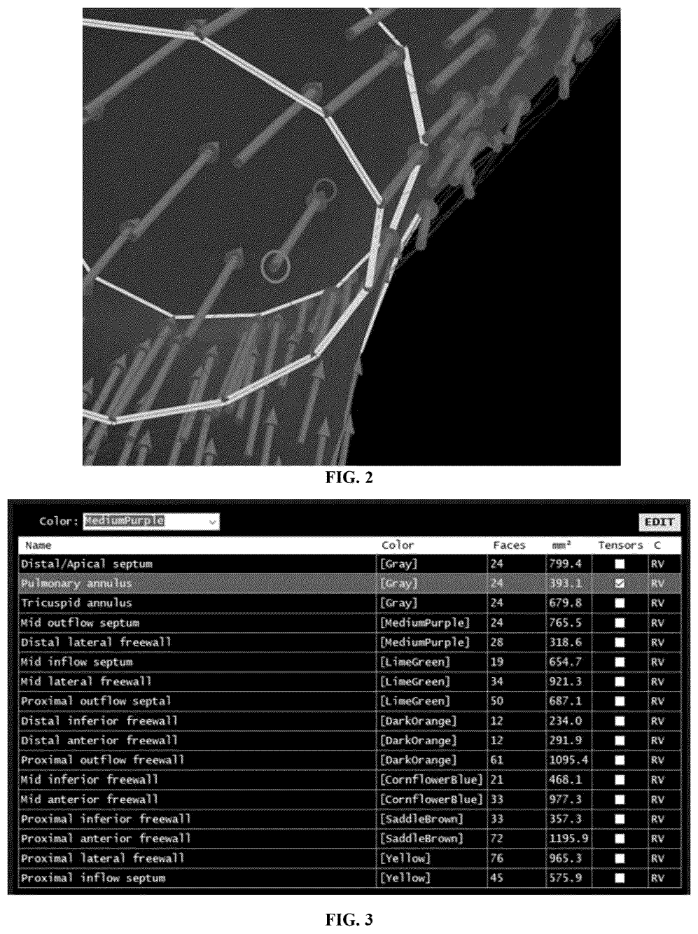

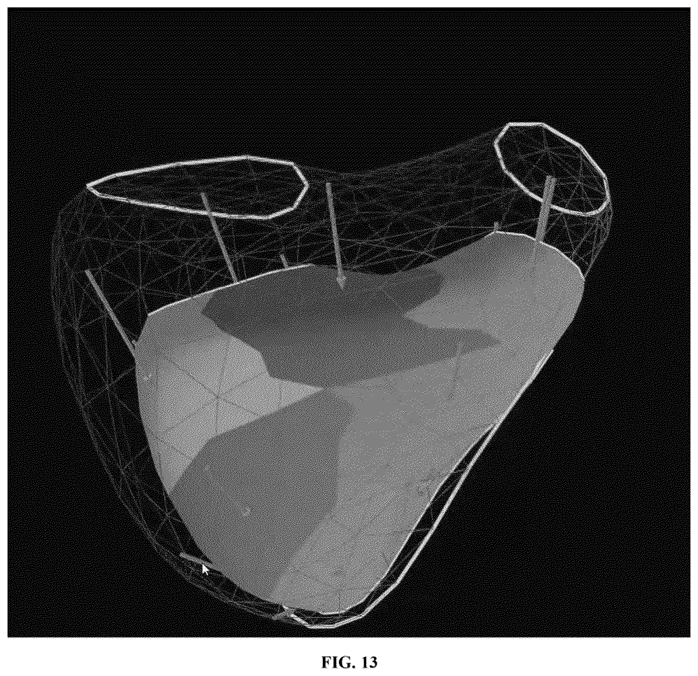

In a preferred embodiment, the present information applies one or more algorithms to two-dimensional image slices of the heart at different times (or frequency) throughout a heart cycle (e.g., between the systolic and diastolic states) for generating three-dimensional models to depict how different chambers of a heart move over time. Heart valves move significantly, and the apex of the heart does not. There may be interest in how the different heart segments move between diastolic and systolic positions of the heart. In a preferred embodiment, the present invention is adapted to quantify the movement. The tensors of the present invention may preferably, but need not necessarily, represent vector movements of the heart wall, as depicted in FIGS. 2 and 9. Persons having ordinary skill in the art will understand that a vector is a mathematical object including a size (or magnitude) and a direction. For example, a vector would be used to show the distance and direction something (e.g., a segment) has moved in. The heart is preferably separated into different segments (defined by the mesh) as shown in FIGS. 3, 4, 5, 10 and 13. In a preferred embodiment, any point on the heart wall may be measured (e.g., by isolating one or more segments). Persons having ordinary skill in the art may appreciate that movement of the heart throughout the heart cycle may be compromised depending on the type and severity of cardiac disease when compared to normal movements of heart.

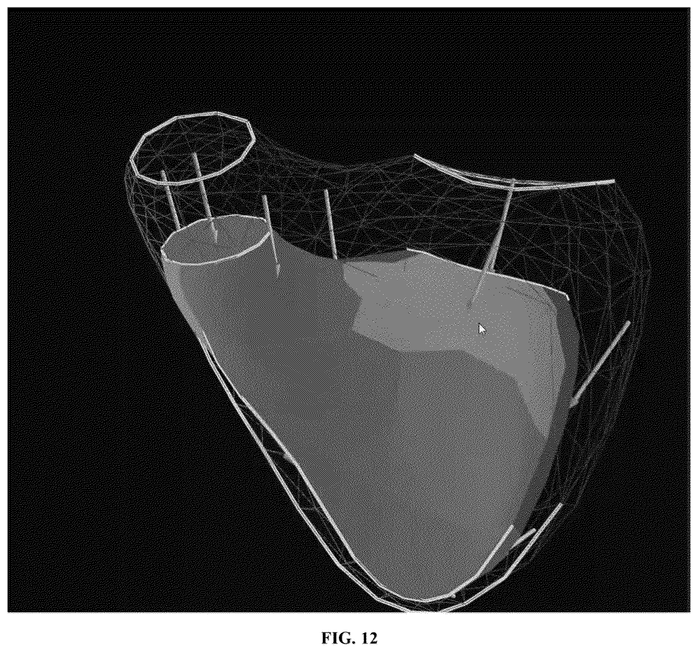

In accordance with a preferred embodiment, tensors may determine how movements are different for diseased hearts when compared to healthy hearts. The one or more segments may preferably, but need not necessarily, provide a quantifiable way to measure the movement of the heart (for adults and children since the shape of the heart changes between childhood and adulthood) for one or more chambers of the heart. As shown in FIGS. 3, 4 and 10, one or more colour-coded segments may be selected. The vectors are depicted as red lines and show movement of the heart chamber when it is at its biggest volume (i.e., diastolic) versus its smallest volume (i.e., systolic) as shown, for example, in FIGS. 2, 9 and 11. In a preferred embodiment, the tensor data includes information associated with sixteen tensors as shown, for example, in FIGS. 3, 4 and 10. The heart segments may preferably, but need not necessarily, facilitate the distillation (or combination) of the number of vectors to follow (or measure) from about 400 to about 16 “tensors” as shown by the yellow arrow(s), for example, in FIGS. 11, 12 and 13. For greater clarity, in a preferred embodiment, one tensor is associated with one or more vectors within a segment. In a preferred embodiment, one arrow (or tensor) depicts the movement of one segment. Regions or segments of the heart are preferably, but need not necessarily, predetermined and set up based on typical uses when imaging the heart. Persons skilled in the art may appreciate that while the about sixteen tensors preferably, but need not necessarily, optimize the amount of data with accuracy of tensor movement, the number of tensors may be greater or less than about sixteen in accordance with the present invention. In a preferred embodiment, two or more segments (alternately “regions”) may be selected for monitoring and/or measurement. Each of the one or more segments may preferably, but need not necessarily, have different names to facilitate monitoring (as shown, for example, in FIGS. 3, 4 and 10). In a preferable embodiment, the heart is segmented (or regionalized) from the base to the apex. The segments may be modified to present more clinically relevant information.

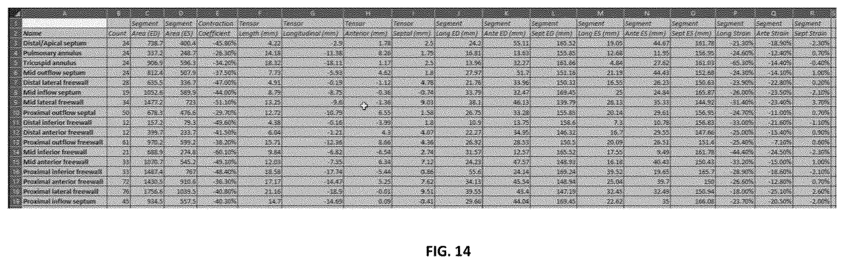

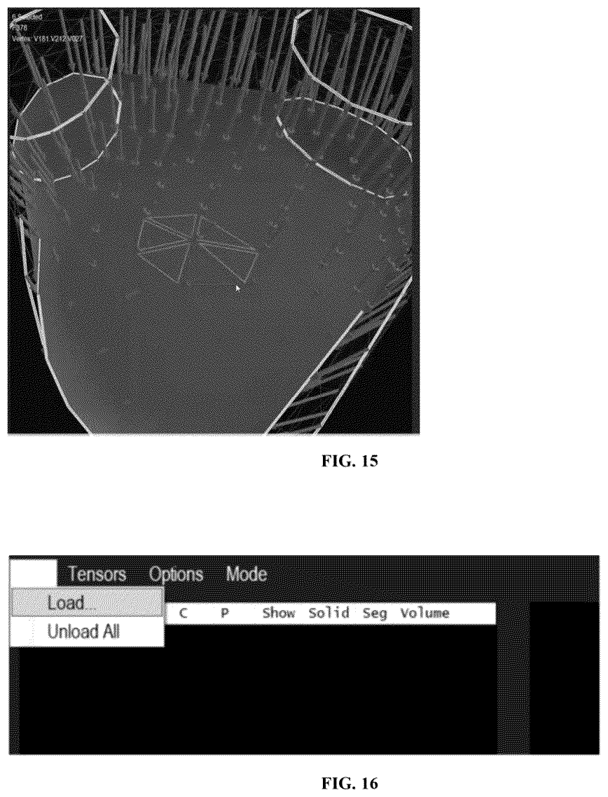

In a preferred embodiment, the tensor data is quantified as shown, for example in FIGS. 3, 4, 10, 14 and 25. The segments, having different names, are shown in FIGS. 3, 4, 10, 14 and 25. The tensor data includes, but is not limited to, surface area, contraction coefficient (% change), length of tensor, other different lengths for different regions, etc. The type and volume of data can be customized depending on the application or clinical use. The length of the tensor (yellow) arrows comprises, or may be split into, the component vectors (red arrows), including: longitudinal (apex to annulus (mitral)), anterior (mitral annulus to aortic annulus), and septal (free wall to septum) directions, and any combination thereof or derivative. In a preferred embodiment, one or more of the segments may be used to obtain surface area and percent change in surface area between the end diastolic (“ED”) and end systolic (“ES”) heart cycle or phase. The present invention preferably generates three-dimensional volumes using two-dimensional scan planes. The three-dimensional meshes preferably, but need not necessarily, provide a 1:1 correlation between the two-dimensional images and three-dimensional model.

In a preferred embodiment, each triangle of the mesh is labeled (e.g., colour coded) to indicate the segment to which it belongs as shown, for example, in FIGS. 4 and 19. In a preferred embodiment, the identical meshes of the diastolic heart and the systolic heart facilitate the generation of tensors. The right ventricle (“RV”), right atrium (“RA”), left atrium (“LA”) and left ventricle (“LV”) chambers, collectively referred to as the heart chambers and each being a heart chamber may be each labeled so the change in sections can be monitored (i.e., by monitoring and/or measuring the same section or segment between the systolic heart and the diastolic heart). The mesh associated with the ES heart may be subtracted from the ED heart (or vice versa) since the labeled triangular meshes of a heart's chamber for ED and ES have the same number of vertices and the vertices match in anatomical features as shown in FIG. 2 The tensors preferably, but need not necessarily, demonstrate the incremental change over time, and/or at any given time, for the movement between the systolic and diastolic phases of the heart cycle. Tensor loads are preferably generated to determine how each section of mesh changes. Given that the meshes' vertices have known locations in 3D space, the tensors have both a magnitude and a direction. In a preferred embodiment, a user (e.g., medical doctor) may customize the sections of interest or expand a section (and associated tensors) for more detailed inspection of a particular portion of the heart. A section (or segment) may be expanded, for example, by including more triangles in the segment.

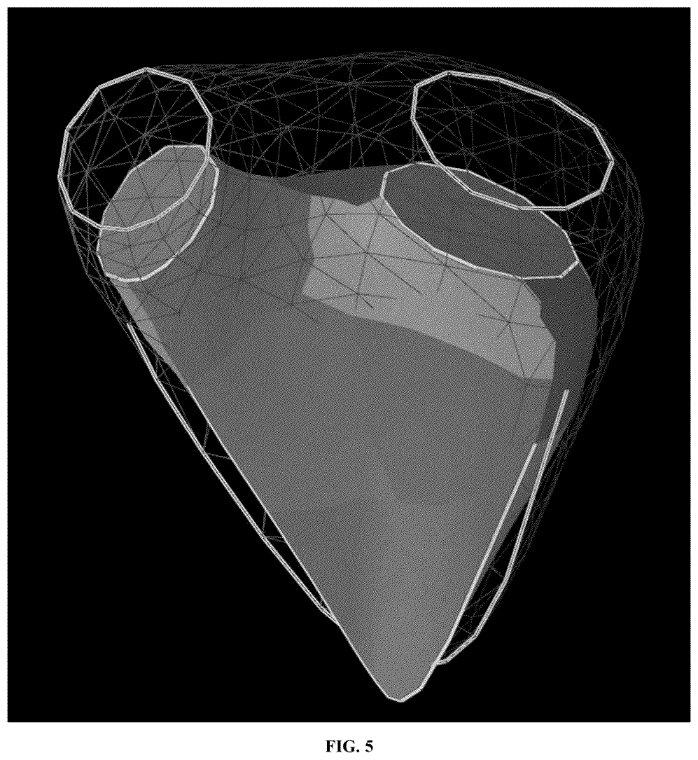

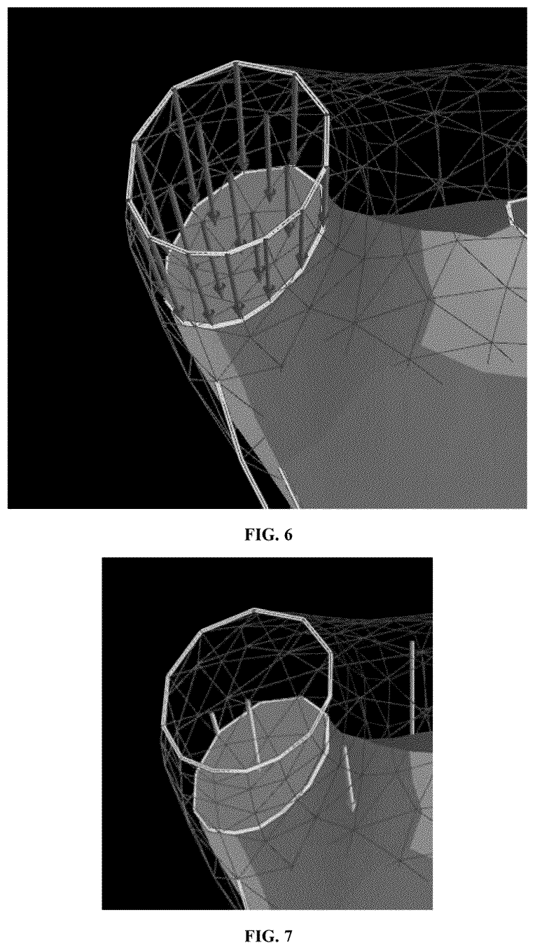

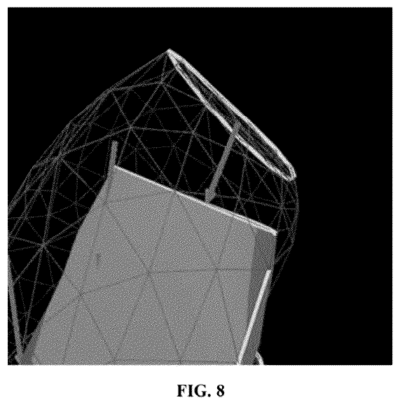

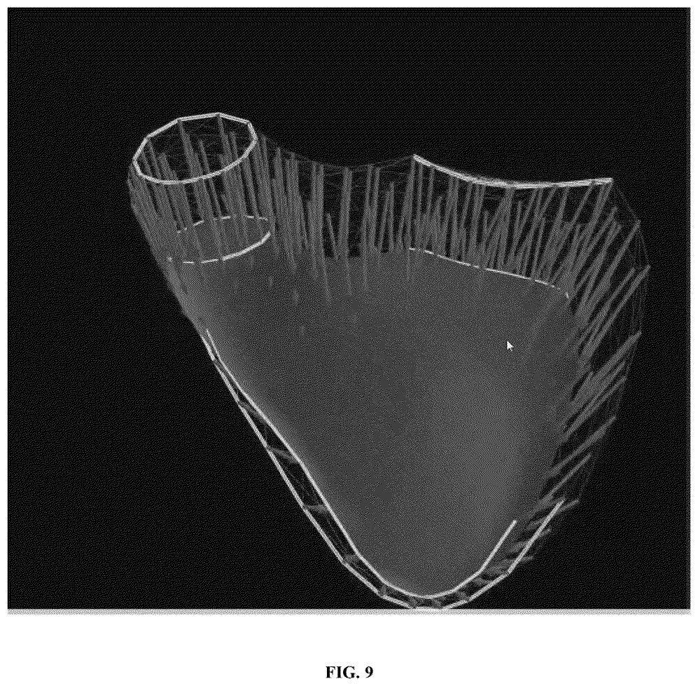

In a preferred embodiment, one or more segments may be created or regions of the meshes may be identified and divided into “segments” as shown in FIG. 3. The segments in the 3D model may preferably, but need not necessarily, be identified as shown in FIGS. 4 and 5. Different contiguous (or non-contiguous) triangles within the mesh may be selected (or grouped together) to generate each segment. Each of the discrete areas or openings (which may preferably, but need not necessarily, be shaped as triangles) forming the mesh may preferably, but need not necessarily, be added or subtracted from each segment to increase/decrease the size of the respective segment (i.e., to customize the segmentation). By creating the segmentation, the tensors may preferably but need not necessarily be grouped by segments, as shown in FIG. 6, to provide an “average” tensor—a representative force for all of the measured vectors—associated with the segment between two or more stages of the heart cycle (depicted by the yellow arrow) as shown in FIGS. 7 and 8. The size of each of the triangles is preferably, but need not necessarily, predetermined. In a preferred embodiment, a user may apply the system, method and/or computer readable medium of the present invention to analyze the heart in, or intermediate to, the ED and/or ES states. The analysis may preferably, but need not necessarily, be performed on the same patient over time and/or of a particular segment of the heart.

In an embodiment of the present invention, the subject (or patient) does not need to be kept still during data collection.

### EXAMPLE

Outputting the meshes to a viewing application of the present invention includes: 1) loading a study in the data collection application with calculations having been performed; 2) exporting the meshes; 3) closing the data collection application; and/or 4) copying and/or viewing the meshes on the device associated with a mesh viewing application.

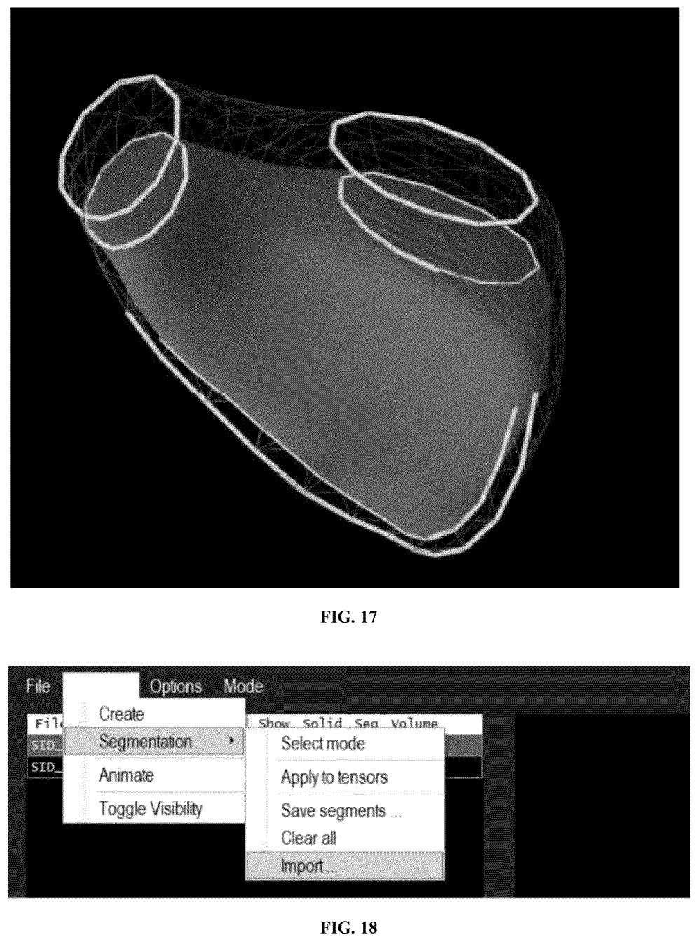

Loading the meshes in the viewing application includes: 1) starting the viewing application; 2) choosing and/or loading the mesh file; 3) selecting both the RV˜ED and RV˜ES. The foregoing steps are depicted in FIGS. 16 and 17.

Selecting tensors, then segmentation, then importation results in segmentation of a heart chamber (e.g., RV), as shown in FIGS. 18 and 19.

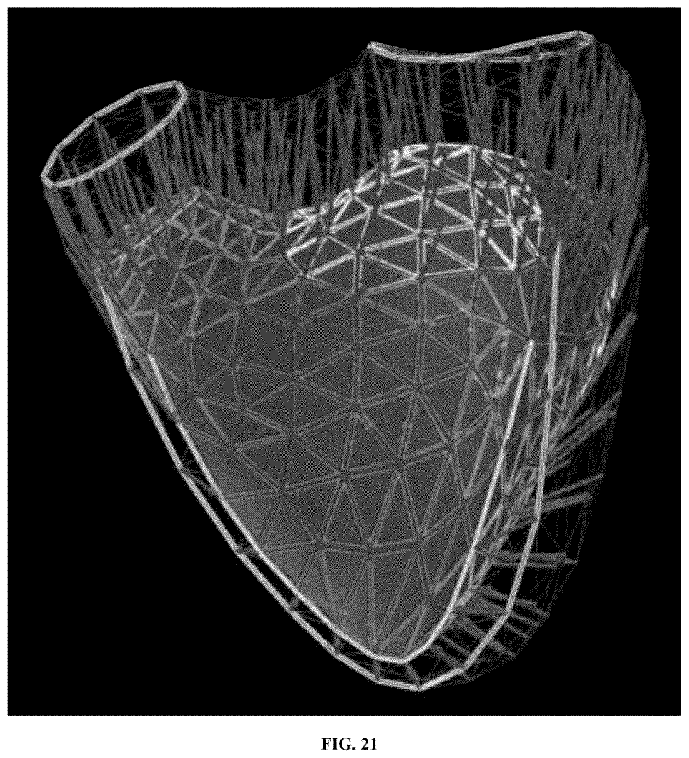

Showing the tensors, includes: 1) selecting both meshes (e.g., ED and ES stages of the heart); and 2) selecting create tensors, as depicted in FIGS. 20 and 21.

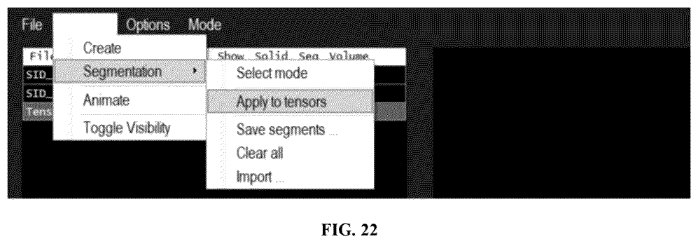

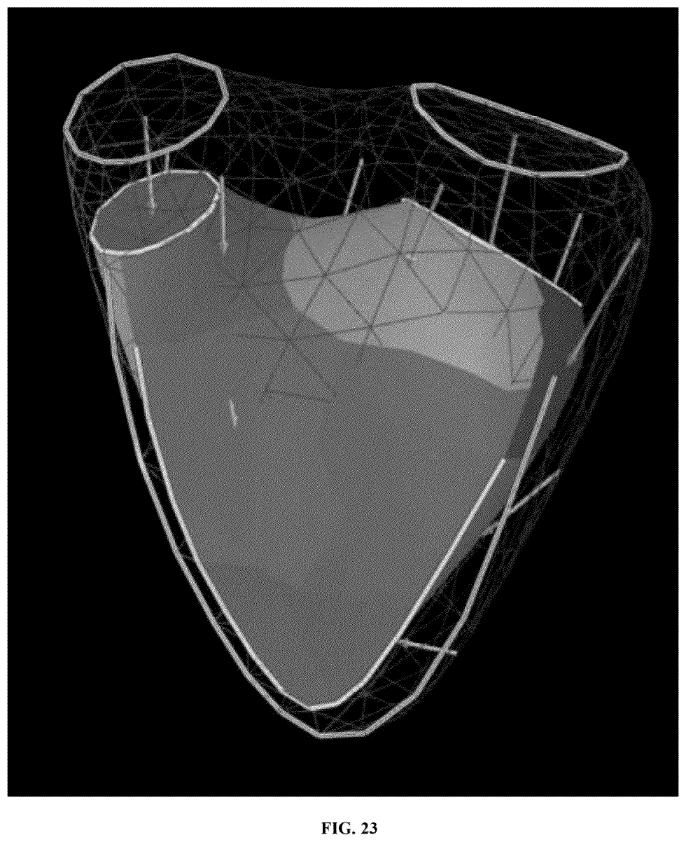

Highlighting the tensors and selecting “segmentation” and “apply to tensors” will generate an average tensor for all the discrete tensors within the segmentation, as shown in FIGS. 22 and 23.

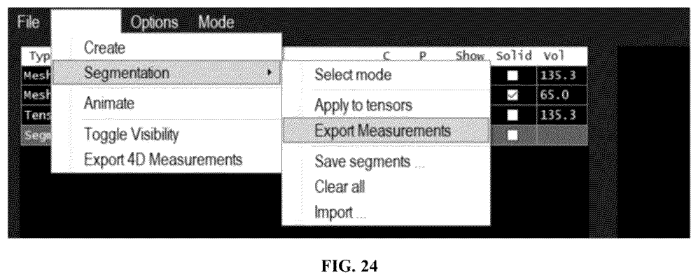

Measurements may be exported by selecting “Tensors”, “Segmentation” and “Export Measurements” as shown in FIG. 24.

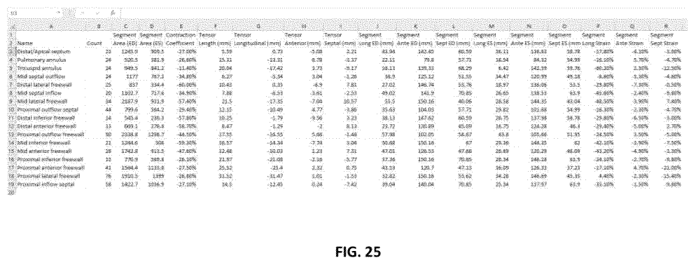

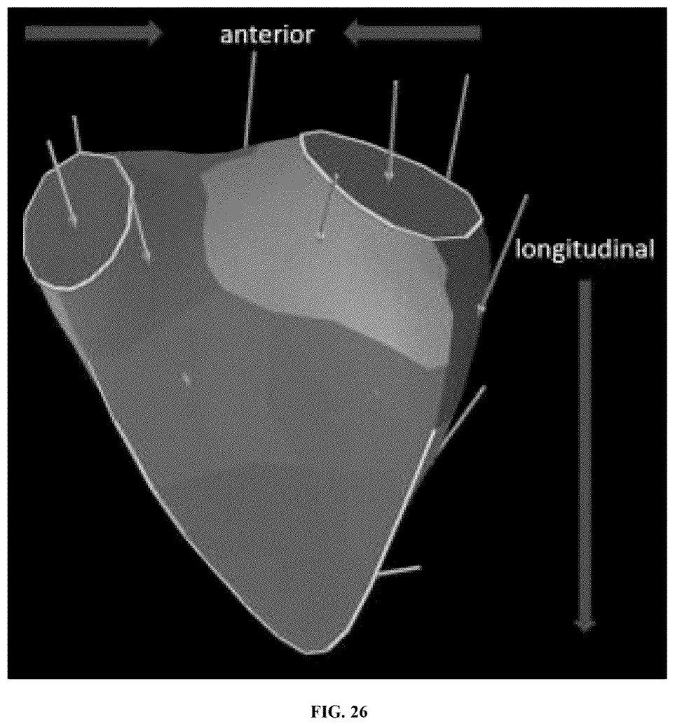

A spreadsheet will be generated based on anterior strain, septal strain and longitudinal strain per segment, as shown in FIGS. 25, 26 and 27.

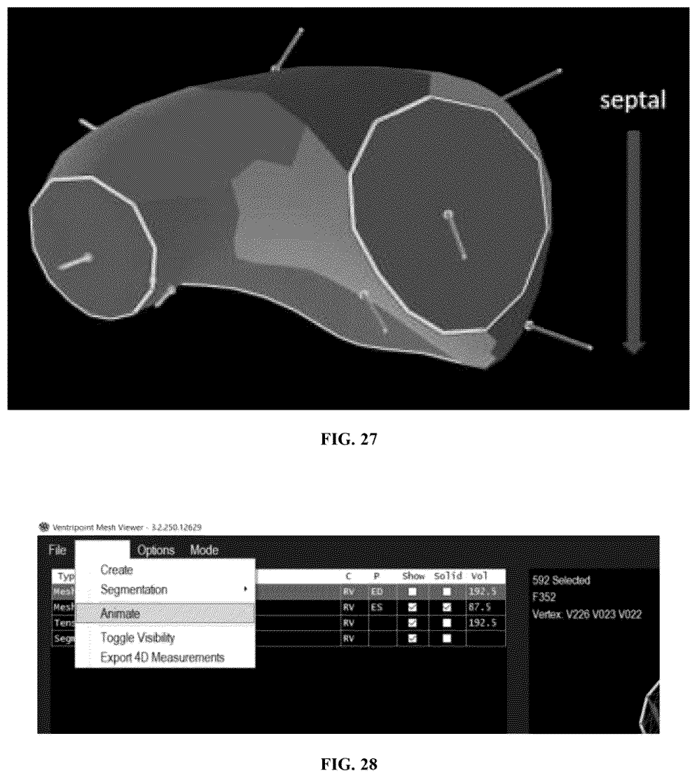

Animation (e.g., ED to ES back to ED) may be displayed by selecting “Highlight tensors row” and “Tensors>Animate” as shown in FIG. 28.

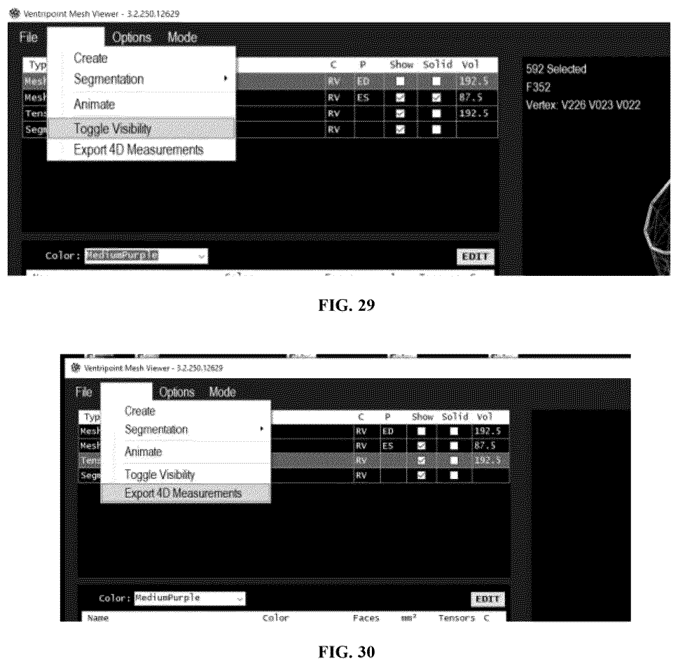

To toggle the tensors during animation, select “Uncheck RV ED Show” and then “Tensors>Toggle Visibility” as shown in FIG. 29.

To obtain the 4D measurements, engage animate and select “Tensors>Export 4D Measurements” as shown in FIG. 30.

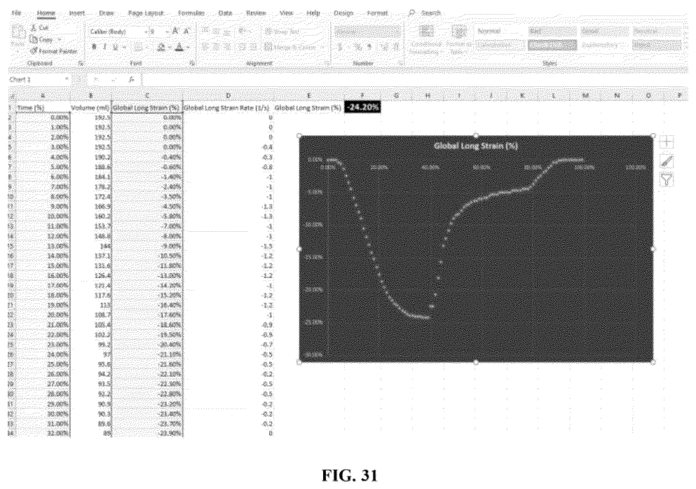

A spreadsheet showing column A (time) graphed against column C (global longitudinal strain)—time is % of cardiac cycle (ED back to ED) is depicted in FIG. 31.

In a preferred embodiment, the two-dimensional image slices are used to generate three-dimensional models of the heart including, but not limited to, systolic and diastolic phases of the heart cycle. The present invention preferably, but need not necessarily, measures the movement of the walls of the heart (including one or more specific chambers of the heart) by tracking segments between the two heart phases.

The present disclosure may be described herein with reference to system architecture, block diagrams and flowchart illustrations of methods, and computer program products according to various aspects of the present disclosure. It may be understood that each functional block of the block diagrams and the flowchart illustrations, and combinations of functional blocks in the block diagrams and flowchart illustrations, respectively, can be implemented by computer program instructions.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flow diagram illustrations support combinations of means for performing the specified functions, combinations of steps for performing the specified functions, and program instruction means for performing the specified functions. It may also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions.

In this disclosure, several terms and abbreviations may be used. The following definitions and descriptions of such terms and abbreviations are provided in greater detail.

It may be further generally understood by a person skilled in the relevant art that the term “downloading” refers to receiving datum or data to a local system from a remote system or to initiate such a datum or data transfer. Examples of a remote systems or clients from which a download might be performed include, but are not limited to, web servers, FTP servers, email servers, or other similar systems. A download can mean either any file that may be offered for downloading or that has been downloaded, or the process of receiving such a file. A person skilled in the relevant art may understand the inverse operation, namely sending of data from a local system to a remote system may be referred to as “uploading”. The data and/or information used according to the present invention may be updated constantly, hourly, daily, weekly, monthly, yearly, etc. depending on the type of data and/or the level of importance inherent in, and/or assigned to, each type of data. Some of the data may preferably be downloaded from the Internet, by satellite networks or other wired or wireless networks.

Elements of the present invention may be implemented with computer systems which are well known in the art. In general, computers include a central processor, system memory, and a system bus that couples various system components including the system memory to the central processor. A system bus may be any of several types of bus structures including a memory bus or memory controller, a peripheral bus, and a local bus using any of a variety of bus architectures. The structure of a system memory may be well known to those skilled in the art and may include a basic input/output system (“BIOS”) stored in a read only memory (“ROM”) and one or more program modules such as operating systems, application programs and program data stored in random access memory (“RAM”). Computers may also include a variety of interface units and drives for reading and writing data. A user of the system can interact with the computer using a variety of input devices, all of which are known to a person skilled in the relevant art.

One skilled in the relevant art would appreciate that the device connections mentioned herein are for illustration purposes only and that any number of possible configurations and selection of peripheral devices could be coupled to the computer system.

Computers can operate in a networked environment using logical connections to one or more remote computers or other devices, such as a server, a router, a network personal computer, a peer device or other common network node, a wireless telephone or wireless personal digital assistant. The computer of the present invention may include a network interface that couples the system bus to a local area network (“LAN”). Networking environments are commonplace in offices, enterprise-wide computer networks and home computer systems. A wide area network (“WAN”), such as the Internet, can also be accessed by a computer, a mobile device or the device.

It may be appreciated that the type of connections contemplated herein are exemplary and other ways of establishing a communications link between computers may be used in accordance with the present invention, including, for example, mobile devices and networks. The existence of any of various well-known protocols, such as TCP/IP, Frame Relay, Ethernet, FTP, HTTP and the like, may be presumed, and computer can be operated in a client-server configuration to permit a user to retrieve and send data to and from a web-based server. Furthermore, any of various conventional web browsers can be used to display and manipulate data in association with a web-based application. In addition, any of various mobile applications (including but not limited to iOS and Android applications) can be used to display and manipulate data.

The operation of the network ready device (i.e., a mobile device) may be controlled by a variety of different program modules, engines, etc. Examples of program modules are routines, algorithms, programs, objects, components, data structures, etc. that perform particular tasks or implement particular abstract data types. It may be understood that the present invention may also be practiced with other computer system configurations, including multiprocessor systems, microprocessor-based or programmable consumer electronics, network PCS, personal computers, minicomputers, mainframe computers, and the like. Furthermore, the invention may also be practiced in distributed computing environments where tasks are performed by remote processing devices that are linked through a communications network. In a distributed computing environment, program modules may be located in both local and remote memory storage devices.

Embodiments of the present invention can be implemented by a software program for processing data through a computer system. It may be understood by a person skilled in the relevant art that the computer system can be a personal computer, mobile device, notebook computer, server computer, mainframe, networked computer (e.g., router), workstation, processor onboard the device and the like. In one embodiment, the computer system includes a processor coupled to a bus and memory storage coupled to the bus. The memory storage can be volatile or non-volatile (i.e., transitory or non-transitory) and can include removable storage media. The computer can also include a display, provision for data input and output, etc. as may be understood by a person skilled in the relevant art.

Some portion of the detailed descriptions that follow are presented in terms of procedures, steps, logic block, processing, and other symbolic representations of operations on data bits that can be performed on computer memory. These descriptions and representations are the means used by those skilled in the data processing arts to most effectively convey the substance of their work to others skilled in the art. A procedure, computer executed step, logic block, process, etc. is here, and generally, conceived to be a self-consistent sequence of operations or instructions leading to a desired result. The operations are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following description, it is appreciated that throughout the present invention, references utilizing terms such as “receiving”, “creating”, “providing”, “communicating” or the like refer to the actions and processes of a computer system, or similar electronic computing device, including an embedded system, that manipulates and transfers data represented as physical (electronic) quantities within the computer system's registers and memories into other data similarly represented as physical quantities within the computer system memories or registers or other such information storage, transmission or display devices.

The present invention is contemplated for use in association with one or more cooperating environments, to afford increased functionality and/or advantageous utilities in association with same. The invention, however, is not so limited.

Naturally, in view of the teachings and disclosures herein, persons having ordinary skill in the art may appreciate that alternate designs and/or embodiments of the invention may be possible (e.g., with substitution of one or more steps, algorithms, processes, features, structures, parts, components, modules, utilities, etc. for others, with alternate relations and/or configurations of steps, algorithms, processes, features, structures, parts, components, modules, utilities, etc.).

Although some of the steps, algorithms, processes, features, structures, parts, components, modules, utilities, relations, configurations, etc. according to the invention are not specifically referenced in association with one another, they may be used, and/or adapted for use, in association therewith.

One or more of the disclosed steps, algorithms, processes, features, structures, parts, components, modules, utilities, relations, configurations, and the like may be implemented in and/or by the invention, on their own, and/or without reference, regard or likewise implementation of one or more of the other disclosed steps, algorithms, processes, features, structures, parts, components, modules, utilities, relations, configurations, and the like, in various permutations and combinations, as may be readily apparent to those skilled in the art, without departing from the pith, marrow, and spirit of the disclosed invention.

While computer-readable storage medium may be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

It may generally be understood by a person skilled in the relevant art that the term “cloud computing” is an information technology model that facilitates ubiquitous access to shared pools of configurable system resources and higher-level services that can be provisioned with minimal management effort, usually over the Internet. Third-party clouds preferably enable organizations to focus on their core businesses instead of allocating resources on computer infrastructure and maintenance.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and software components, or only in software.

In the present description, numerous details are set forth. It will be apparent, however, to one of ordinary skill in the art having the benefit of this disclosure, that the present disclosure may be practiced without these specific details. In some instances, well-known structures and devices are shown in block diagram form, rather than in detail, in order to avoid obscuring the present disclosure.

The present disclosure also relates to an apparatus for performing the operations herein. This apparatus may be specially constructed for the required purposes, or it may include a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (“ROMs”), random access memories (“RAMs”), EPROMs, EEPROMs, magnetic or optical cards, or any type of media suitable for storing electronic instructions.

The foregoing description has been presented for the purpose of illustration and maybe not intended to be exhaustive or to limit the invention to the precise form disclosed. Other modifications, variations and alterations are possible in light of the above teaching and may be apparent to those skilled in the art, and may be used in the design and manufacture of other embodiments according to the present invention. It may be intended the scope of the invention be limited not by this description but only by the claims forming a part of this application and/or any patent issuing therefrom.

1. A method for measuring a change in magnitude and/or direction of movement of a heart chamber during a heart cycle, wherein the method comprises the steps of:
(a) collecting a plurality of 2D images associated with the heart chamber during the heart cycle;
(b) generating a plurality of segments of the heart chamber during the heart cycle using segment generation and assigning each of the plurality of segments to a surface of an anatomical position associated with the heart chamber; and
(c) quantifying an angle and a displacement of each of the plurality of segments assigned to the surface of the anatomical position from an end diastolic to an end systolic phases of the heart cycle to determine the magnitude and/or direction of movement of each of the plurality of segments assigned to the surface of the anatomical position associated with the heart chamber during the heart cycle.

2. The method of claim 1 further comprising:
(d) determining a plurality of vectors for each of the plurality of segments based on the change in magnitude and direction; and
(e) determining a tensor data based on the plurality of vectors for each of the segments to demonstrate an incremental change over time of the movement of the heart chamber between the systolic and diastolic phases of the heart cycle.

3. The method of claim 2 wherein the tensor data include surface area, contraction coefficient, and length of tensor which represents volume and function of the heart chamber during the heart cycle.

4. The method of claim 2 wherein the tensor data comprises the data set out in Table 1.

5. The method of claim 3 wherein images are made using 2D ultrasound, 3D ultrasound or MRI.

6. The method of claim 5 wherein the tracking of the plurality of segments measures the movement of the walls of the heart chamber.

7. The method of claim 6 wherein the heart chamber is selected from a RV, RA, LV, LA.

8. The method of claim 7 wherein the tracking of the plurality of segments provides information on the viability or health of the heart chamber.

9. The method of claim 8 further comprising:
(f) using a movement algorithm to track the plurality of segments in the ED volume versus the ES volume as determined by a user selecting specific anatomical landmarks of the heart chamber as an input to a KBR algorithm;
(g) calculating the displacement of the matching plurality of segments to determine the movement of the segments over the heart cycle.

10. A method for measuring a change in magnitude and/or direction of a heart chamber during a heart cycle, wherein the method comprises the steps of:
(a) collecting a plurality of images associated with the heart chamber during the heart cycle; and
(b) operating one or more processors to: (i) generate two or more three-dimensional models of the chamber during the heart cycle using the plurality of images; (ii) overlay a mesh on each of the two or more three-dimensional models of the chamber; (iii) divide the mesh into a plurality of segments; (iv) measure the change in magnitude and/or direction of each of the plurality of segments during the heart cycle; (v) determine one or more vectors for each of the segments based on the change in magnitude and/or direction; and/or (vi) determine one or more tensors based on the one or more vectors for each of the segments;
wherein the tensors represent volume and function of the heart chamber during the heart cycle.

11. The method of claim 10 wherein step (b) further comprises a user selecting a plurality of triangles within the mesh to generate a single segment within the mesh.

12. The method of claim 10 wherein the triangles are contiguous or non-contiguous triangles.
